(19) United States
(12) Patent Application Publication
(10) Pub. No.: US 2026/0315262 A1
(43) Pub. Date: Oct. 15, 2026

(54) The Breastfeeding Clip

(71) Applicant: Ashley Marie Coleman, Oceanside, CA (US)

(72) Inventor: Ashley Marie Coleman, Oceanside, CA (US)

(21) Appl. No.: 19/644,629

(22) Filed: Apr. 10, 2026

Related U.S. Application Data
(60) Provisional application No. 63/787,887, filed on Apr. 12, 2025.

(51) Int. Cl. A61F 5/03

(52) U.S. Cl. CPC A61F 5/03

(57) ABSTRACT

A clip device for positioning breast tissue during breastfeeding is disclosed. The Clip includes an upper member and a lower member connected by a connecting mechanism and a biasing element configured to move the members between an open position and a closed position. The Clip is configured to hold and reposition breast tissue to create a space between the breast tissue and a baby's nose during feeding and to provide support in place of manual hand positioning. The device may further facilitate access to a nipple and assist with latch and milk transfer during feeding, including in cases of inverted or downward-facing nipples. The Clip allows for hands-free operation while maintaining breast tissue in a desired position.

Field of the invention: This invention relates to a device, referred to herein as The Clip, used to assist mothers and infants during breastfeeding by positioning breast tissue to improve latch, milk transfer, and comfort.

Background/Problem: Many breastfeeding mothers (especially those with larger breasts) have to use their own hands to hold breast tissue in U-, C-, or V-shaped positions, where portions of the hand are on top of the tissue holding it down and back, and other portions support tissue from below. Mothers use this hold during feedings to:
1. Prevent breast tissue from covering the baby's nose
2. Improve latch by helping the baby grasp sufficient tissue
3. Address inverted or downward-facing nipples to enable effective latch and milk transfer

The Clip addresses many breastfeeding challenges by providing:
1. A safe space for babies, creating a gap between breast tissue and nose during feeding
2. Hands-free operation for the mother while supporting all breastfeeding needs
3. Proper manipulation of breast tissue to help the baby latch more effectively, resulting in efficient milk transfer
4. Assistance in achieving proper latch, which can reduce nipple soreness

Mothers typically breastfeed 8-12 times per day, and during each feed, must manually maintain the C-, U-, or V-shaped hold. This can be difficult, especially when multitasking, tired, or caring for other children. Maintaining the hold over time can also cause hand fatigue. Breastfeeding already involves multiple challenges, and adding additional obstacles—such as holding tissue manually or ensuring the baby's safety—can negatively impact the experience and may lead to decreased breastfeeding, stress, or anxiety.

The Clip addresses these problems by positioning breast tissue as desired during feeding, preventing obstruction of the baby's face, and relieving the mother from manual support. By mimicking natural U-, V-, and C-shaped holds, the Clip can improve latch, provide hands free relief for the mother, enhance milk transfer, and reduce nipple soreness. Overall, the Clip improves multiple aspects of the breastfeeding experience, addressing common challenges for both mother and baby while allowing hands-free feeding.

### BACKGROUND/PRIOR ART

A search of existing products indicates that there are no devices currently available that specifically assist mothers in maintaining breast tissue in a desired position during breastfeeding while providing hands-free support. Many mothers, particularly those with larger breasts, must use their hands to hold tissue back to prevent obstruction of the baby's nose and to improve latch. Mothers with small or medium-sized breasts may use manual holds to help the baby latch effectively or to address inverted nipples.

The Clip addresses this need by mimicking the natural U-, V-, and C-shaped holds mothers use, providing hands-free support and improving milk transfer, latch efficiency, and comfort. Its design makes it suitable for mothers of all breast shapes and sizes.

### BRIEFLY DESCRIBE INVENTION

The Clip is a device designed to support breastfeeding by positioning and manipulating breast tissue during feeding. It includes an upper and lower arm connected by a hinge and spring mechanism, which allows the arms to open and close to accommodate breast tissue of varying sizes and shapes. The device can be made from materials such as plastic, metal, or other suitable biocompatible materials.

During use, the Clip is placed on the breast to maintain the desired tissue position, creating a safe space between the baby's nose and breast tissue. This prevents obstruction of the airway and facilitates proper latch. By mimicking the natural U-, V-, and C-shaped holds that mothers often use manually, the Clip provides hands-free support, reducing hand fatigue during frequent breastfeeding sessions.

The adjustable nature of the Clip allows it to accommodate different breast shapes, sizes, and nipple orientations, including inverted or downward-facing nipples. By holding the tissue in the desired position, the device can improve latch efficiency, optimize milk transfer, and potentially reduce nipple soreness caused by improper latch.

The Clip may be used during each breastfeeding session and can remain in place for the duration of feeding or as needed to support the mother and infant. The design ensures safety, comfort, and effectiveness, while allowing the mother to engage in other activities or care for other children during feeding sessions.

Currently, no commercially available devices provide a hands-free method to support breast tissue while addressing these challenges for mothers of all breast sizes. The Clip solves this problem by replacing the mother's hand, allowing for safe, efficient, and hands-free breastfeeding while improving latch and milk transfer.

### SUMMARY OF THE INVENTION

The Clip is designed to hold and manipulate breast tissue in a desired position during breastfeeding. Key benefits include:
Maintaining a gap between the baby's nose and breast tissue for safe feeding.
Mimicking the natural U-, C-, or V-shaped hold used by mothers to improve latch and milk transfer.
Supporting mothers with inverted or downward-facing nipples to facilitate better attachment.
Reducing hand fatigue and mental stress during feeding.
Allowing hands-free breastfeeding to improve the overall experience for both mother and baby.

The Clip can be used by mothers of all breast sizes and shapes, providing a universal solution to common breastfeeding challenges.

### BRIEF DESCRIPTION OF THE DRAWINGS

FIG. 1 is a front view of the clip device in a closed position.

FIG. 2 is a front view of the clip device in an open position.

FIG. 3 is a side view of the clip device positioned on breast tissue.

FIG. 4 is a front view of the clip device positioned on breast tissue.

FIG. 5 illustrates alternative configurations of the clip device, including C-shaped, U-shaped, and V-shaped configurations.

FIG. 6 illustrates the clip device in use during breastfeeding.

FIG. 7 illustrates an embodiment of the clip device including adjustable and/or interchangeable components.

### DETAILED DESCRIPTION OF THE INVENTION

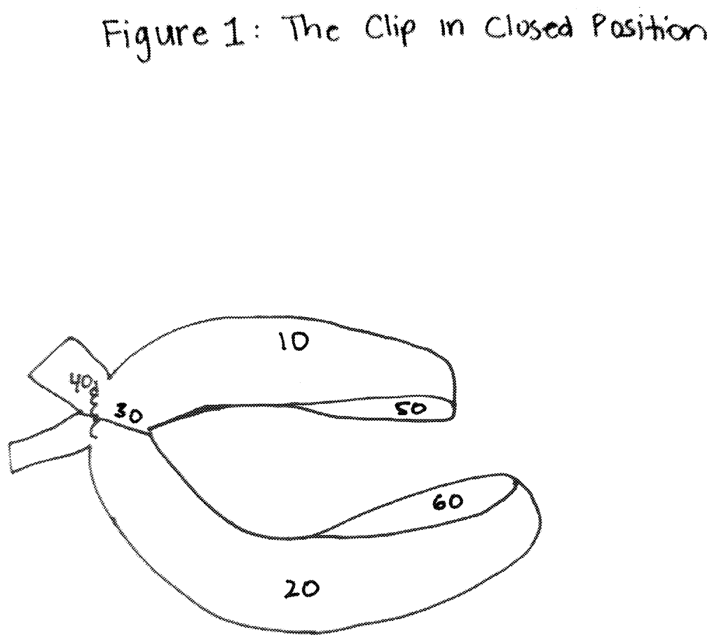

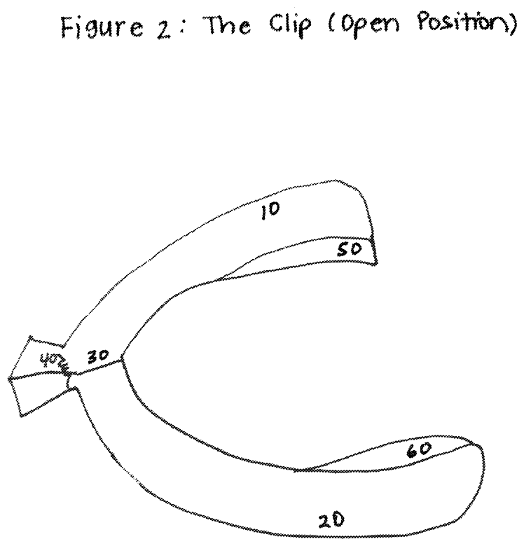

As shown in FIGS. 1-2, the clip device includes an upper member (10) and a lower member (20) connected by a connecting mechanism (30) and a biasing element (40), which allows the clip device to move between an open position and a closed position.

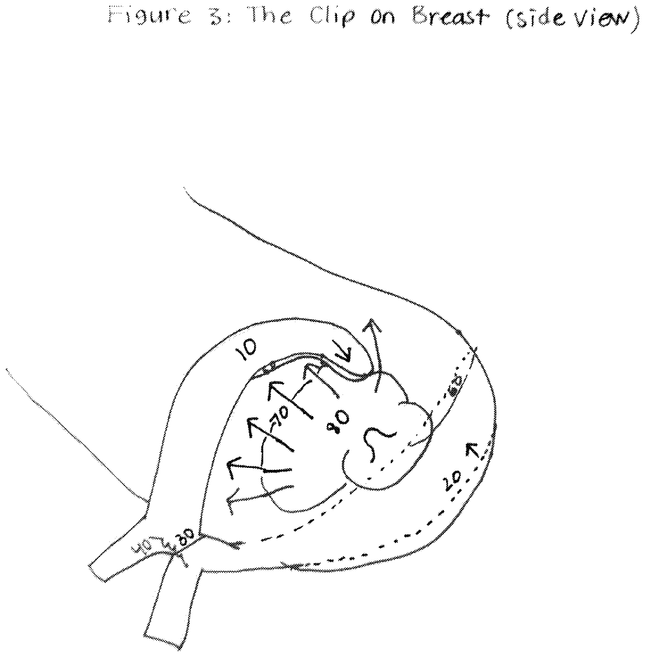

As shown in FIG. 3, the upper member (10) and lower member (20) cooperate to reposition breast tissue (70), creating a space (80) within the breast tissue during use. The upper gripping area (50) and lower gripping area (60) are configured to grip the breast tissue and assist in maintaining positioning of the clip device.

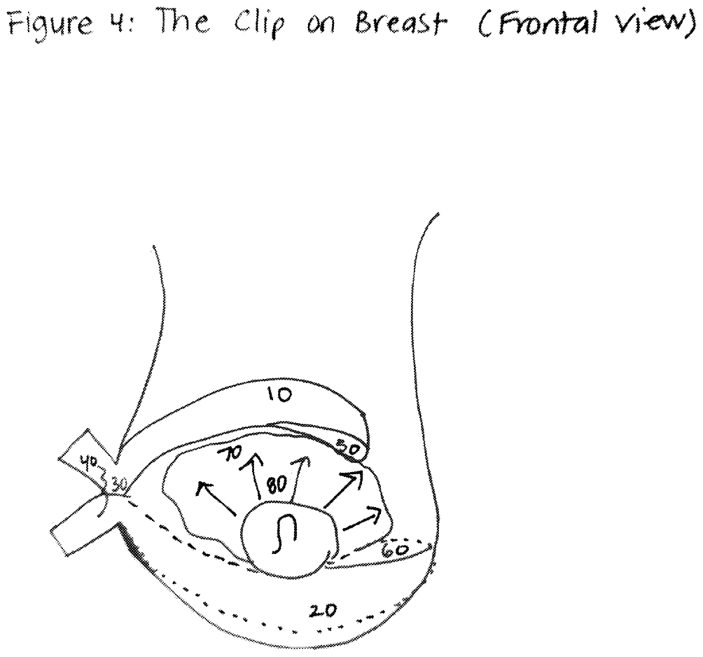

As shown in FIG. 4, the clip device is positioned across breast tissue (70) to maintain placement during use. The upper gripping area (50), located on the upper member (10), and the lower gripping area (60), located on the lower member (20), are configured to contact and grip the breast tissue to assist in maintaining positioning of the clip device, with the lower portion of the clip positioned beneath the breast tissue as indicated by dashed lines.

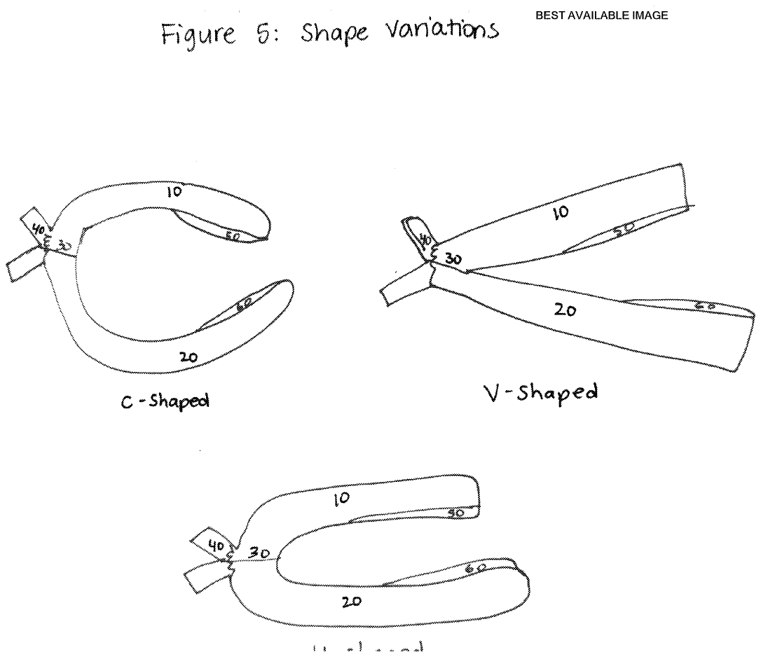

As shown in FIG. 5, the clip device may be formed in different configurations, including C-shaped, U-shaped, and V-shaped forms.

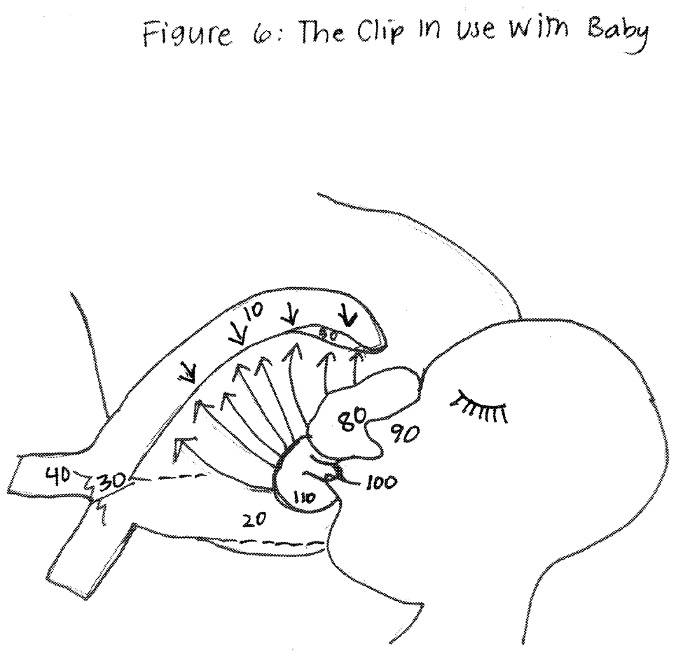

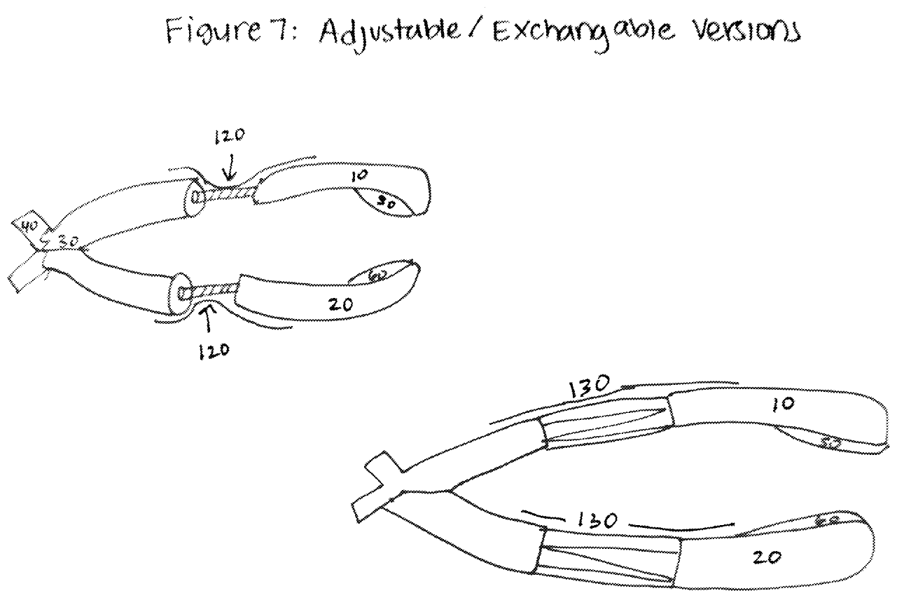

As shown in FIG. 6, the clip device is used during breastfeeding to maintain spacing (80) between breast tissue (70) and a baby's nose (90), with the baby's mouth (100) positioned for feeding, and to facilitate access to a nipple. The clip device is configured to reposition breast tissue, including moving upper breast tissue downward and rearward, to maintain the breast tissue in a desired position during feeding and to assist with latch. As shown in FIG. 7, the clip device may include adjustable or interchangeable components, including an adjustable or sliding portion (130) and interchangeable arms (120).

### Parts, Components, Features

### Parts

Upper portion/arm of clip
Lower portion/arm of clip
Spring/hinge mechanism to open and close the clip
Clip base components (upper portion/arm, lower portion/arm, spring hinge, base material)
Clip cover (medical, silicone, grade rubber-safe for skin contact)
Clip outer cover (soft material safe on skin and easy to wash)
May include extendable features for the upper and lower portion (sliding mechanism allowing clip arms to extend)
May include changing of arms to fit mothers preferred shape/size
The connecting mechanism may include a hinge having a pivot member, such as a rod, pin, or axle, about which the upper and lower members rotate.

### Components

Base: Molded plastic, metal, or other suitable material forming the structure of the Clip (could be fixed or flexible for needed adjustments) .
Upper Clip: Shorter portion designed to hold the upper breast tissue down.
Lower Clip: Longer or wider portion supporting the bottom breast tissue.
Spring Mechanism: Allows the Clip to open and close, holding breast tissue securely.
Cover for Base: Medical-grade silicone rubber or other skin-safe material, washable.
Outer Cover with padding: Soft material safe on skin that is Machine washable and covers/provides padding for soft safe skin contact.
Mechanisms: Open/close of clip using springs, Sliding or Additional/Removable Upper and lower portions of the clip to accommodate different breast sizes/shapes.
Outer Cover: Soft, hypoallergenic fabric (cotton, bamboo, silk) for comfort; machine washable.
Grips: On silicone or fabric covers to hold the Clip securely against breast tissue.
In certain embodiments, at least one of the upper or lower portions of The Clip may be flexible, deformable, or segmented such that it conforms to the natural curvature of the breast during use

### Features

### Features on Clip or Cover

Grip on silicone mold at end of clip to better grip to breast tissue
Grip on cloth material covering base to better grip to breast tissue
Pinch end of clip to open using spring loaded action
Release end of the clip that results in it closing and holding its place when you let go.
Material that is safe on skin, hypoallergenic
May include padding to cloth cover for increased comfort
Will adjust as needed for better functionality and use (ex: silicone mold cover may require grips to help grip to breast tissue), may have a slidable piece on clip to accommodate all breast types and allow customers to slide top clip to desired spot to push down tissue.
May include Sliding mechanism to extend upper/lower portion/arms
May include pull out and swap feature for desired arms
In certain embodiments, at least one of the upper or lower portions of The Clip may be flexible, deformable, or segmented such that it conforms to the natural curvature of the breast during use
The biasing element of The Clip may include a spring, flexible hinge, or other mechanism configured to provide a controlled and gentle clamping force sufficient to maintain positioning of breast tissue without excessive compression or discomfort. In certain embodiments, the biasing element may be selected or configured to provide varying levels of force.
May be designed to resemble a hand

### How it Works

1. Pinch the ends of the Clip to open the upper and lower portions.
   a. Adjust clip as needed to fit breast shape/size. If sliding or swapping arms on clip, do so now.
2. Slide the bottom portion under the breast and the top portion over the breast, positioning it in the desired area.
3. The upper portion of The Clip is positioned above the nipple on the upper breast tissue at a desired location (to keep upper breast tissue down). This tissue is held down to address the issues described above (prevent babies face. nostrils from being covered, address inverted, down facing nipples, improve latch/milk transfer, hands free).
4. Release the Clip to hold the breast tissue in place.
5. Adjust clip parts (make upper or lower portions longer or shorter to accommodate shape of breast or size if adjustments are still needed)
6. Breastfeed hands-free while the Clip maintains safe breast tissue positioning, improves latch, and enhances milk transfer.
7. To remove or open the clip, pinch the Clip ends to open and slide it off the breast.
8. Once removed, wash the silicone or fabric covers as needed for hygiene.

### Variations and Modifications

Size of The Clip: May have Small, medium, large to fit different breast sizes, shapes (round, raindrop, symmetrical, teardrop, east-west, conical, bell shaped), and tissue densities (fatty, fibrous, mixed) or Nipple Placement (high, low, inverted) . This may be addressed with a sliding mechanism, adjustable upper and lower portions or changing out upper and lower portions.
Size of the upper and lower portions/arms of The Clip: The Clip may be different sizes (top vs bottom, etc). The smaller clip on top (could be 2 inches, 4 inches, 6 inches, etc). The bottom clip may be 5 inches, 10 inches, etc. The bottom clip may be narrow or may have two connected parts for wider base or 1 part for wider base providing more breast support. (inches may vary)
Clip Shapes: U-, C-, V-shaped or resemble mothers fingers/hand parts in the design of the product to mimic natural hand holds. The Clip may be more round to fit around the breast, may be more flat to hold tissue down, may be curved (u shaped) or a mix. C shaped (larger breasts), U Shaped or V shaped (medium to smaller breasts). Maybe a mix.
Clip designs: Formation of top and bottom clips (bottom clip may be narrow, wider, longer, whereas top clip may be wider, shorter, narrow (resembling finger/thumb pushing down), may make bottom wider to support more breast (resembling hand/multiple fingers holding).
What holds The Clip Together: The spring/hinge component holds the upper and lower portions. arms together and provides function to open and close the clip to adjust and fit on breast tissue as desired. The sliding mechanism or the pull out ability of the upper/lower arms allow the length/width to be adjusted as needed.
Adjustable Components: Top/bottom portion/s may slide, adjust or be able to be pulled out and replaced with ideal top or bottom shape to better fit breast anatomy.
Materials: Base can be plastic or metal; covers can be silicone or soft fabric; springs or mechanisms can vary for optimal function.
Grip Options: Added grips or padding on silicone/fabric for secure placement and comfort.
Clamp Force of The Clip: The biasing element of The Clip may include a spring, flexible hinge, or other mechanism configured to provide a controlled and gentle clamping force sufficient to maintain positioning of breast tissue without excessive compression or discomfort. In certain embodiments, the biasing element may be selected or configured to provide varying levels of force.

### Advantages/Novelty

No known products currently address the hands-free support of breast tissue for breastfeeding.
The Clip improves latch and milk transfer, especially for mothers with inverted or downward-facing nipples.
Universally adaptable for all breast sizes and shapes.
Reduces hand fatigue, mental stress, and safety concerns during feeding.

### Steps/Process of Use

1. Pinch The Clip's ends to open.
2. Place the bottom portion under the breast.
3. Position the top portion above the nipple or on upper breast tissue as desired (resulting in the nipple pointing upward and enough space between breast tissue and baby's nose).
4. Release Clip to hold tissue in place.
5. Breastfeed hands-free with improved safety and efficiency.
6. Pinch ends to open and remove Clip after feeding.
7. Wash silicone/fabric covers for next use.

### Modifications

May modify material used for, base, base cover, material cover, grips, clip or spring mechanism etc. to best support breastfeeding mothers. May modify sizes to best fit mother and will modify breast clip details in any way needed to address needs from mothers and improve overall product use, functionality, etc.

### Additional Embodiments/Variations

In certain embodiments, The Clip may include adjustable features to accommodate a variety of breast shapes, sizes, and tissue characteristics. For example, the upper and/or lower portions of The Clip may include sliding, telescoping, or extendable arms that allow the length of each portion to be increased or decreased to fit the user's anatomy. In other embodiments, the upper and/or lower portions may be interchangeable or modular, allowing different sized or shaped components to be attached to a base structure.

Additionally, portions of The Clip may include flexible, pivoting, or multi-segmented structures configured to conform to the natural curvature of the breast. These structures may allow The Clip to approximate U-, V-, or C-shaped configurations, thereby mimicking natural hand positioning used during breastfeeding. The Clip may further include malleable or semi-rigid materials, such as flexible polymers or internally supported structures, which allow the device to adapt to individual breast contours while maintaining sufficient support to hold tissue in a desired position.

These variations allow The Clip to provide customized support and positioning while maintaining its primary function of enabling safe, efficient, and hands-free breastfeeding.

### Materials Used

A variety of materials may be used to form The Clip, including but not limited to plastics, metals, or composite materials for the base structure. The Clip may include a covering made from medical-grade silicone or other skin-safe materials. An optional outer covering may be formed from soft, hypoallergenic fabrics. A spring or other biasing element may be used to operate the opening and closing of the Clip. Grip elements may be included to help secure the Clip in position on breast tissue.

1. A clip device for positioning breast tissue during breastfeeding, comprising:
an upper member configured to engage an upper portion of breast tissue;
a lower member configured to engage a lower portion of breast tissue;
a connecting mechanism joining the upper member and the lower member and allowing movement between an open position and a closed position; and
a biasing element configured to apply force to maintain the upper member and the lower member in the closed position, wherein the upper member and the lower member are configured to reposition breast tissue to create a space between the breast tissue and a baby's nose during feeding and to facilitate access to a nipple.

2. The clip device of claim 1, wherein the biasing element comprises a spring.

3. The clip device of claim 1, wherein the connecting mechanism comprises a hinge.

4. The clip device of claim 1, wherein the upper member is shorter than the lower member.

5. The clip device of claim 1, wherein at least one of the upper member or the lower member includes a skin-contacting surface formed from silicone or another flexible material safe for skin.

6. The clip device of claim 1, wherein at least one of the upper member or the lower member includes a grip surface configured to maintain position on breast tissue.

7. The clip device of claim 1, wherein at least one of the upper member or the lower member is adjustable in length.

8. The clip device of claim 7, wherein the adjustable member includes a sliding or telescoping mechanism.

9. The clip device of claim 1, wherein the curvature approximates a C-shaped, U-shaped, or V-shaped configuration.

10. The clip device of claim 1, wherein at least one of the upper member or the lower member is removable or interchangeable.

11. The clip device of claim 1, wherein the biasing element is configured to provide a controlled clamping force sufficient to maintain positioning of breast tissue while reducing excessive compression.

12. The clip device of claim 1, wherein the upper member and the lower member are further configured to facilitate access to a nipple.

13. The clip device of claim 1, wherein positioning of the breast tissue facilitates latch and milk transfer during feeding.

14. The clip device of claim 1, wherein the upper member and the lower member are configured to position breast tissue in a manner corresponding to a manual hand hold used during breastfeeding.
